(19) United States
(12) Patent Application Publication
(10) Pub. No.: US 2005/0085700 A1
(43) Pub. Date: Apr. 21, 2005

(54) Method and apparatus for dynamic motion analysis

(76) Inventors: Christopher Neil Sharp, West Glamorgan (GB); Hillary May Ann, West Glamorgan (GB)

Correspondence Address: STITES & HARBISON PLLC, 1199 NORTH FAIRFAX STREET, SUITE 900, ALEXANDRIA, VA, 22314, US

(21) Appl. No.: 10/493,915

(22) PCT Filed: Oct. 24, 2001
(86) PCT No.: PCT/GB01/04725

(51) Int. Cl. G06F 19/00; G01N 33/48; G01N 33/50

(52) U.S. Cl. 600/300; 702/19

(57) ABSTRACT

In order to determine a physical or physiological condition, a series of images of a human subject in motion are captured (1, 2, 3). At least one image (19) is then displayed, and the user selects from a displayed category list (8) an appropriate category to represent the subject's condition. The category list represents categories of physical or physiological conditions. A list of treatments for treating the subject (5) may be displayed, as may a list of possible treatment products, e.g. footwear. Where the treatment products are footwear, there may be a foot measuring device (10) and other information about the subject (5) may be stored so as to assist the user in selection of the appropriate footwear products.

### BACKGROUND OF THE INVENTION

### FIELD OF THE INVENTION

The present invention relates to a method and apparatus for use in the analysis of the dynamics of a human subject, and particularly, though not exclusively, for use in the identification in a human subject of one or more physical or physiological conditions apparent in, or associated with, the walking or running deportment of that subject.

Summary of the Prior Art

The identification and analysis of physical or physiological “conditions” in the feet of human subjects currently relies upon established techniques of podiatry or chiropody. Here, physical ailments or other conditions are often identified when the human subject is static. The subject sits or lies on a bed and a therapist or other trained practitioner manipulates the subject's feet to discover the degrees of movement of the ankles, arches and toe areas so as to determine the variation of the subject from the theoretical normal. In some instances, measurements of the lower limbs would be taken. Since the subject sits or lies during such manipulation, this is a semi weight bearing investigation. The disadvantage of such techniques lie in the difficulty of identifying ailments or conditions which only become apparent, or are more easily identifiable, when the subject is viewed when walking or running. In particular, certain conditions or ailments may be best identified at particular instants within the walking or running cycle of a subject. In the known techniques, the subject may be asked to stand and walk a few steps. The therapist/practitioner then watches the subject and observes weight bearing movement with the naked eye.

### SUMMARY OF THE INVENTION

Where a subject is viewed in a walking or running state, it is generally not possible to closely and repeatedly scrutinise the dynamics of e.g. the subject's feet at a given instant in time due to the very fact that the subject is in motion and such instants are inherently transient. Consequently, the ability to accurately and/or quickly analyse the physical condition of e.g. the subject's feet is hampered.

The present invention aims to overcome at least some of the deficiencies of the prior art.

At its most general, the invention proposes to capture one or more images of at least a subjects feet, in a standing, walking or running state, from one or more different perspectives, and to display the image(s) concurrently with information (e.g. a list) representing a number categories under which physical conditions identified from the images may be categorised during analysis of the images.

In this way, the invention aims to enable the accurate yet rapid and simple identification and categorisation of physical or physiological conditions in e.g. the feet of a subject via an analysis of the deportment of that subject. By viewing the subject images concurrently with suitable categorising information, the viewer is able to identify and analyse physical or physiological conditions in the subject, and to simultaneously categorise those conditions by selecting suitable categories from the concurrently displayed category information.

In a first of its aspects, the invention provides a method of categorising a human subject comprising the steps of:
capturing a series of images of said human subject in motion in the form of image data;
providing a category list representing a plurality of categories of possible physical or physiological conditions in said human subject;
inputting to an image display apparatus said captured image data and said category list;
displaying via said image display apparatus at least one image from said series of captured images concurrently with said category list;
selecting from said category list one or more of said categories with which to categorise a respective one or more physical or physiological condition(s) identified in said human subject from said displayed at least one image.

The image(s) may be a still image or a sequence of still images, or a moving image sequence which may be “frozen” to provide still images as required by the viewer. Preferably, a plurality of such images are simultaneously displayed, each from a different perspective but captured at the same instant or over the same time interval. However, images from different instants, or covering different or partially overlapping time intervals may be simultaneously displayed.

The images of the subject preferably include one or more front-view images of the feet of the subject in a standing or walking or running state. Preferably concurrently displayed are images including one or more rear-view images of the feet of the human subject in a standing or walking or running state. Thus, a front and rear view of the dynamics of the subject may be viewed together. More preferably, the displayed images include one or more rear-view images of at least the lower spinal area of the human subject in a standing or walking or running state. Other views of the body of the subject may be displayed instead or in addition.

The images may be captured by one or more digital cameras for example, although still cameras, video recorders/players or scanners may be used. The camera(s) are preferably connected to, or connectable to, a computer terminal which receives the image data and stores (at least temporarily) that data for display or displays it immediately via the visual display unit (VDU) of the computer terminal. Thus, in this example, the aforementioned display apparatus is the VDU of the computer terminal, but other display apparatus may be used for displaying the images whether digital or otherwise. For example, a TV monitor could be used or images could even be printed.

In order to obtain the images it is preferable that the human subject is caused to be in a standing or walking or running state on a treadmill, and the images of the subject are preferably captured while the subject is in such a state. This enables the dynamics of the subject to be imaged in one convenient location, it obviated the need to move the image capturing apparatus with the subject or to track the subject. It may also be possible for the images to be captured in other situations, such as on a running or athletic track.

This enables the imaging apparatus to be kept in a fixed position such that any movement of the subject shown by the captured images will be due substantially only to the motion of the subject and not to motion of the imaging device(s). The speed of walking/running motion can be easily controlled by suitably adjusting the speed of the treadmill, and the perspective(s)/view(s) provided by imaging apparatus may be adjusted to suit the user's needs.

In addition to displaying a category list concurrently with the captured images, other related information may be concurrently displayed therewith. For example, information regarding a number of possible treatments for physical conditions/ailments listed in the category list may be displayed. This information may be in the form of a list of possible treatments, the list being retrievable by the user as and when required or being automatically presented upon selection by the user of one of (or a predetermined number of) the categories from the category list. Thus, preferably, the method also includes the steps of:
displaying via said display apparatus a treatment list representing one or more treatments each associated with one or more of said one or more categories in said category list; and,
selecting from said treatment list one or more treatments.

Furthermore, information regarding a number of treatment products for effecting the treatment of the physical conditions/ailments listed in the category list may be displayed. This information may be in the form of a list of possible treatment products, e.g. appropriate footwear, the list being retrievable by the user as and when required or being automatically presented upon selection by the user of one of (or a predetermined number of) the treatments from the treatment list. Thus, preferably, the method includes the steps of:
displaying via said display apparatus a treatment product list representing a plurality of treatment products each associated with one or more of said treatments in said treatment list; and,
selecting from said treatment product list one or more treatment products.

Additional data may be employed in analysing the human subject and preferably such data includes person data of the physical characteristics of the subject, such as height, age, weight etc. Thus, the method preferably includes the step of acquiring subject data representing physical characteristics of the human subject, inputting the acquired data to the display apparatus, and displaying at least some of the acquired data thereby.

Preferably, the acquired data includes at least data relating to the dimensions of the subject's feet, and this data may be acquired from a suitable device connected to the display apparatus (e.g. to a VDU via a computer terminal) for direct input of that data.

The data representing the category list, treatment list, treatment product list and subject data are preferably stored in a database and are respectively retrieved therefrom prior to being displayed. Preferably, each of the category list, treatment list, treatment product list and subject data are stored in a separate respective database.

Whilst the display of the image, and the display of the treatment and/or product list, may be substantially immediate on the capturing of the image data, this is not essential. It is possible for the image data to be stored e.g. on tape, DVD or CD Rom for subsequent display. It is also possible for the image data to be transmitted (e.g. via a modem) to a remote site for display.

### BRIEF DESCRIPTION OF THE DRAWINGS

There now follows a detailed, but non-limiting, illustration of the present invention by way of an example, with reference to the following drawings:

FIG. 1 illustrates a schematic representation of apparatus for the dynamic analysis of the motion of a human subject.

### DETAILED DESCRIPTION

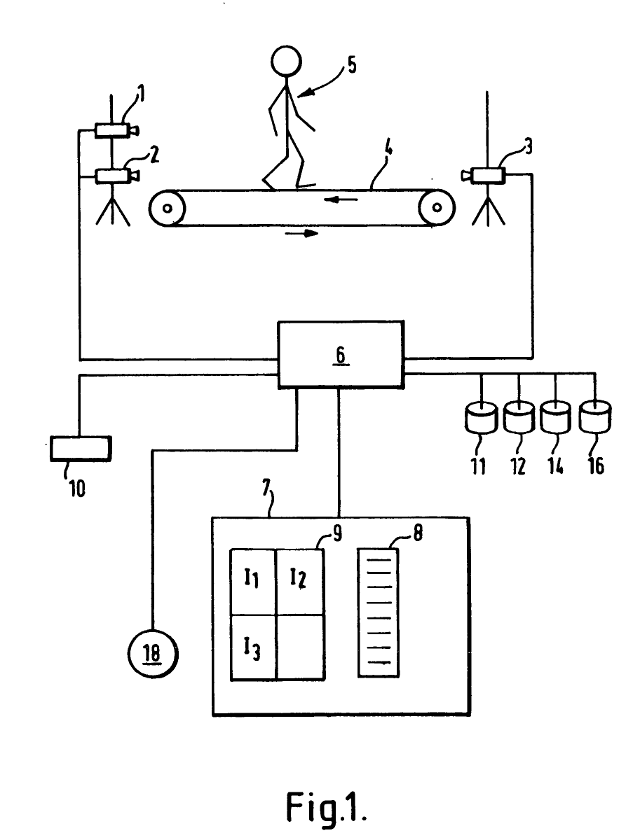

In FIG. 1, an array of three digital cameras, 1, 2 and 3, are arrayed about an operating treadmill 4 upon which a human subject 5 located in a walking state sufficient to keep him upon the treadmill. A first digital camera, camera 1, is positioned so as to capture digital moving images if the rear spinal area at the base of the back of the subject 5, while second and third digital cameras, cameras 2 and 3, are arranged to capture moving digital images of the rear and front view of the feet of the subject respectively.

Each of the three digital cameras 1, 2 and 3 are connected to a computer terminal 6 in such a way as to permit the transfer of digital image data thereto. Thus, digital image data captured by the three cameras is continuously input to a computer terminal 6 as the subject 5 walks upon the treadmill 5. Images of that walking motion are captured by the three cameras as viewed from the front and rear of the subject's feet (illustrating footstrike), and from the base of the subjects back.

The computer terminal 6 subsequently controls the visual display unit (VDU) 7 so as to display the views provided by the images captured by the three camera array. The three views of the subject 5 are presented in a regular display format 9 comprising a first view in the form of moving images I₁, I₂ and I₃ provided by the first, second and third digital cameras 1, 2 and 3 respectively.

Concurrently displayed with the captured image display 9 is a display 8 comprising a list representing a plurality of predetermined categories of possible physical or physiological conditions in the subject 5.

Data representing the displayed category list is stored in a database 11 and input to the computer terminal 6 for display at the VDU 7 concurrently with the captured images 9 either automatically or at the request of the user.

The user is then able to select from the category list 8 one or more categories with which to categorise a respective one or more physical or physiological condition(s) identified in the subject 5, the selection being made on the basis of an analysis of the displayed images 9. Selection may be made by, for example, operating user controls 18 connected to the computer terminal 6. The user controls may be in the form of known peripheral computer controls such as a “mouse” for controlling the position of a control cursor on the VDU screen 7, or cursor control keys on a computer keyboard.

Any number of categories may be chosen from the category list. In order to assist the analysis of the images 9 of the subject 5, additional information may be displayed with the category list and with the captured images 9. This other information may be personal information related to the subject 5 (e.g. age, sex); information conveying certain physical characteristics of the subject (e.g. weight, height); medical history; the demands of the subject's normal physical activities, if any for example, the positional demands of different sporting activities may affect the treatment desired. This information is stored in a database 12 connected to the computer terminal 6 and is retrievable thereby upon the command of the user, for display at the VDU 7 of the computer.

Information regarding a number of possible treatments for physical conditions/ailments listed in the category list may be displayed. This information may be in the form of a list of possible treatments, the list being retrievable by the user as and when required or being automatically presented upon selection by the user of one of (or a predetermined number of) the categories from the category list. The treatment list (not shown) would be presented in a manner similar to that of category list 8, and would present one or more treatments each associated with one or more of the categories in the category list.

Any number of treatments in the list may be selected by the user, the data representing the treatment list being stored in a database 14, and input to computer the terminal 6 for display at the VDU 7 of the computer at the command of the user. Upon selecting a (or a predetermined number of) treatments from the treatment list, information regarding a number of treatment products for effecting the treatment of the physical conditions/ailments listed in the category list may be displayed. This information may be in the form of a list of possible treatment products and displayed in the manner of display 8. The list may be retrievable by the user as and when required or may be automatically presented upon selection by the user of one of (or a predetermined number of) the treatments from the treatment list. The treatment products are, for example, footwear and the list may be split into categories representing use, foot function, joint type, manufacturer, style and cost.

In the treatment product list, the treatment products are each associated with one or more of said treatments in said treatment list, and the user may select from the treatment product list any number of treatment products.

Moreover, particularly where the treatment products are footwear, it is possible for the treatment product list to be divided into different categories to enable the user more readily to select from that list. Footwear may be divided into last types and length and width fittings, but may also be divided into the different purposes to which the footwear are to be put, such as running, walking, or other sport-related activities as have previously been mentioned, the user may be presented with information indicating the subject's normal physical activities, or sports activities of the subject, and the division of the list into different categories then facilitate the selection of the appropriate product.

Additional data may be input to the computer terminal 6 so as to assist in the analysis of the dynamic motion of the subject 5. Such data includes data relating to the dimensions of the subject's feet, and this data being acquired from a suitable foot measuring device 10 connected to the VDU 7 via the computer terminal for direct input of that data and subsequent display. The data from the foot measuring device 10 may be converted to footwear sizing in any appropriate sizing systems. Sizing may include both length and width information. The data may also include information about the subject's normal physical activities and any sporting activity.

Many variations on the above embodiment are possible within the scope of the present invention. For example, the subject may be marked with dots or lines so that the movements can be more clearly seen, and thus aid analysis and diagnosis. It is possible to store the images for such retrieval, and possibly comparison with current images. This enables improvements, deteriorations, physical changes through injury, or orthotic wearing to be identified. It is also then possible to take measurements when the subject is barefoot, and when they are wearing different types of footwear, to observe the different effects. This also enables the effects on gait of different footwear, bodyloadings, etc. to be observed.

Moreover, the selected treatment, or treatment product may be printed by the apparatus, together with appropriate personal information of the subject, as a permanent record of the selection.

1. A method of categorizing a human subject comprising the steps of:
capturing a series of images of said human subject in motion in the form of image data;
providing a category list representing a plurality of categories of possible physical or physiological conditions in said human subject;
inputting to an image display apparatus said captured image data and said category list;
displaying via said image display apparatus at least one image from said series of captured images concurrently with said category list;
selecting from said category list one or more of said categories with which to categorize a respective one or more physical or physiological condition (s) identified in said human subject from said displayed at least one image.

2. A method according to claim 1, wherein the method includes the steps of:
displaying via said display apparatus a treatment list representing one or more treatments each associated with one or more of said one or more categories in said category list; and,
selecting from said treatment list one or more treatments.

3. A method according to claim 2, wherein the method includes the steps of:
displaying via said display apparatus a treatment product list representing a plurality of treatment products each associated with one or more of said treatments in said treatment list; and,
selecting from said treatment product list one or more treatment products.

4. A method according to claim 1, wherein the treatment products are footwear.

5. A method according to claim 1, wherein the method includes the step of acquiring subject data representing physical characteristics of the feet of the human subject, inputting said acquired data to said display apparatus, and displaying said acquired data thereby.

6. A method according to any claim 1, wherein the method includes the step of inputting to said display apparatus subject data representing predetermined characteristics of the human subject, and displaying at least some of said subject data via said display apparatus.

7. A method according to claim 2, wherein data representing said treatment list is stored in a database and is retrieved therefrom prior to said displaying thereof.

8. A method according to claim 3, wherein data representing said treatment product list is stored in a database and is retrieved therefrom prior to said displaying thereof.

9. A method according to claim 6 wherein said subject data is stored in a database and is retrieved therefrom prior to said displaying thereof.

10. A method according to claim 1, wherein said images include one or more front-view images of the feet of the human subject in a standing or walking or running state.

11. A method according to claim 1 wherein, said images include one or more rear-view images of the feet of the human subject in a standing or walking or running state.

12. A method according to claim 1 wherein, said images include one or more rear-view images-of at least the lower spinal area of the human subject in a standing or walking or running state.

13. A method according to claim 1, wherein said human subject is caused to be in a standing or walking or running state on a treadmill, and said images of said human subject include images captured while said human subject is in such a state.

14. Apparatus for use in categorizing a human subject, the apparatus comprising:
an image capturer for capturing in the form of image data a series of images of said human subject in motion;
a data store for storing a category list representing a plurality of categories of possible physical or physiological conditions in said human subject;
an image display for receiving said image data from said image capturer and for receiving said category list from said data store, and for displaying said at least one of said images concurrently with said category list so as to permit the selecting from said category list one or more of said categories with which to categorize a respective one or more physical or physiological condition (s) identified in said human subject from said displayed at least one image.

15. Apparatus according to claim 14, further including a data store for storing a treatment list representing one or more treatments each associated with one or more of said one or more categories in said category list, said image display being operable to display said treatment list.

16. Apparatus according to claim 15, further including a data store for storing a treatment product list representing one or more treatment products each associated with one or more of said one or more treatments in said treatment list, said image display being operable to display said treatment product list.

17. Apparatus according to claim 14, further including foot measuring means for acquiring subject data representing physical characteristics of the feet of the human subject, and for inputting said acquired data to said display apparatus for display thereby.

18. Apparatus according to claim 14, further comprising a data store for storing subject data representing predetermined physical characteristics of the human subject, said image display being operable to display at least some of said subject data.

19. Apparatus according to claim 15, further including a database in which said treatment list is stored.

20. Apparatus according to claim 16 further including a database in which said treatment product list is stored.

21. Apparatus according to claim 18 further comprising a database in which said subject data is stored.

22. Apparatus according to claim 14 wherein said image capturer is operable to capture one or more front-view images of the feet of the human subject in a standing or walking or running state.

23. Apparatus according to claim 14 wherein, said image capturer is operable to capture one or more rear-view images of the feet of the human subject in a standing or walking or running state.

24. Apparatus according to claim 14 wherein, said image capturer is operable to capture one or more rear-view images of at least the lower spinal area of the human subject in a standing or walking or running state.

25. Apparatus according to claim 14 further including a treadmill wherein, and said image capturer is operable to capture images of said human subject while said human subject is upon said treadmill and in a standing or walking or running state.
